(19) United States
(12) Patent Application Publication
(10) Pub. No.: US 2005/0288582 A1
(43) Pub. Date: Dec. 29, 2005

(54) Micro medical-ultrasonic endoscopic OCT probe

(76) Inventors: Daoyin Yu, Tianjin (CN); Tieying Zhou, Beijing (CN); Ping Xue, Beijing (CN); Xiaodong Chen, Tianjin (CN); Kaili Jiang, Beijing (CN)

Correspondence Address: Liangang (Mark) Ye;Wong Cabello Lutsch Rutherford & Brucculeri, LLP, Suite 600, 20333 SH249, Houston, TX, 77070, US

(21) Appl. No.: 11/149,671

(22) Filed: Jun. 10, 2005

(30) Foreign Application Priority Data
Jun. 28, 2004 (CN) 2004100197463

(51) Int. Cl. A61B 8/00

(52) U.S. Cl. 600/437

(57) ABSTRACT

A micro medical-ultrasonic endoscopic OCT probe comprising: a micro ultrasonic motor stator for connecting a friction layer and a magnetic rotor; an ultrasonic transducer and a prism being adhered to a rotor respectively; an acoustic couplant for immersing the ultrasonic transducer; an OCT imaging system consisting of prism, grim lens and fiber. The present invention can not only observe the pathologic changes on the surface of mucosa through endoscope, but can also obtain the histological tomogram of an organ through OCT scan and ultrasonic scan, thus broadening the diagnosis range and increasing the diagnosis ability of endoscopes. The probe of this invention is driven by a micro motor directly mounted on the front end of the probe, and does not need soft wires. Compared with the existing technology, its lifespan is greatly expanded.

### CROSS REFERENCES TO RELATED APPLICATIONS

This application claims priority from a Chinese patent application, serial number 2004100197463, filed on Jun. 28, 2004 with the same title, by the same inventors, which is incorporated herein by reference.

### FIELD OF INVENTION

This invention relates to a medical instrument, more particularly, to a micro medical-ultrasonic OCT (Optical Coherence Tomography) probe which is used through endoscopes.

### BACKGROUND OF INVENTION

In the diagnosis of pathologic changes, diagnosing deceases by just relying on morphologic changes on the surface of tissue is subjective and has its limitations. An endoscopic ultrasonic OCT imaging system is capable of getting a clear and precise tomogram of organ tissue through both ultrasonic scan and OCT scan, and therefore can provide a physicians with more objective evidence for diagnosis.

Meanwhile, by putting the micro probe into the body cavity through the biopsy orifice of a medical endoscope, not only can the physicians observe the pathologic changes on the surface of mucosa through endoscope, but can also get the histological tomogram of an organ through OCT scan and ultrasonic scan, thus expanding the diagnosis range and increasing the diagnosis ability of the endoscope. As a result, its advantage in diagnosis is well recognized by physicians.

Limited by the dimension of biopsy channel of medical endoscope, the design of a probe is difficult. For example, JP2002153472-A/Fuji Photo Film Co. Ltd. (Hayashi Katsume). -2002.05.28, discloses an imaging diagnosis device which can get OCT and ultrasonic images simultaneously. Its ultrasonic transducer, prism and endoscope are all of hard-tube, and it uses a rear-mounted coreless motor to drive the scan. Its principle is described below.

As shown in FIG. 6, a probe 10a provided with an ultrasonic wave transducer 51 and an OCT scanning part 140 in the inside is inserted to the forceps port of an endoscope and the inside of the colon of a patient is observed. Ultrasonic wave signals are oscillated by an ultrasonic wave signal processor 50, an object is irradiated with ultrasonic waves from the ultrasonic wave transducer 51 on the basis of the signals and measurement is performed. Simultaneously, the light source of an OCT device is driven, the object is irradiated with signal light Ls and the measurement is performed. Further, the probe 10a is rotated by driving a center-less motor 20 and radial scanning is performed. On the basis of information obtained by the scanning, the ultrasonic images and the OCT images are acquired.

Although the device can be inserted into a body through the biopsy channel of a medical endoscope, it cannot be used in soft-tube systems because the endoscope itself is of hard-tube.

### SUMMARY OF THE INVENTION

The objection of present invention is to resolve the problem mentioned above, and to provide a micro medical-ultrasonic OCT probe which can go through an endoscope so that it can be used in soft-tube systems.

The invention provides a micro medical-ultrasonic endoscopic OCT probe comprising:
a micro ultrasonic motor stator for connecting a friction layer and a magnetic rotor;
an ultrasonic transducer and a prism being adhered to a rotor respectively;
an acoustic couplant for immersing the ultrasonic transducer;
an OCT imaging system consisting of prism, grim lens and fiber.

### BRIEF DESCRIPTION OF THE DRAWINGS

A full understanding of the invention can be gained from the following description of the preferred embodiments when read in conjunction with the accompanying drawings in which:

FIG. 1 shows a micro medical-ultrasonic OCT probe operated through endoscope scanning. According to the invention;

FIG. 2 is a structure diagram of the micro probe in embodiment 1;

FIG. 3 is a structure diagram of the micro probe in embodiment 2;

FIG. 4 is a diagram of digestive tract endoscopic handle and biopsy orifice;

FIG. 5 is a structure diagram of a digestive tract endoscope with a micro ultrasonic OCT probe;

FIG. 6 is a structure diagram of prior art.

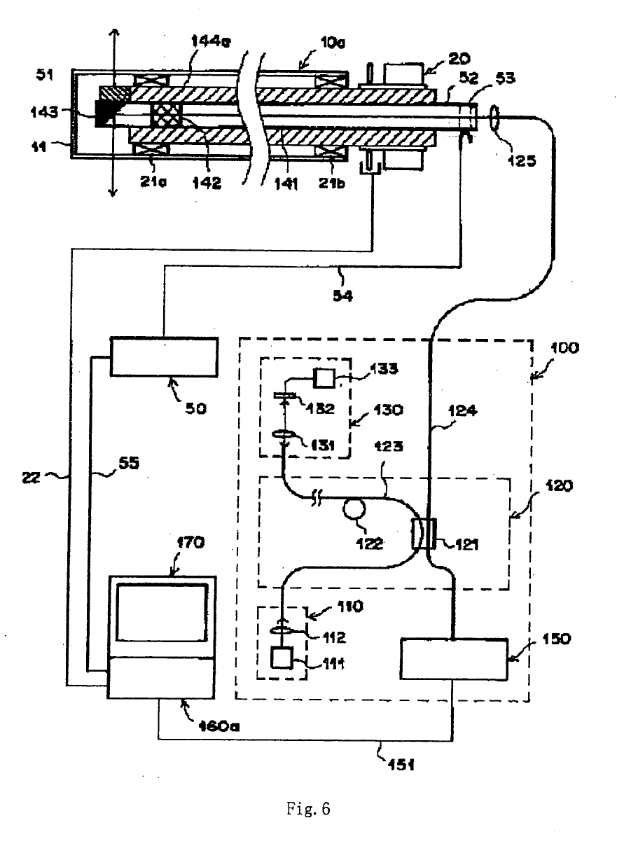

### DESCRIPTION OF THE PREFERRED EMBODIMENTS

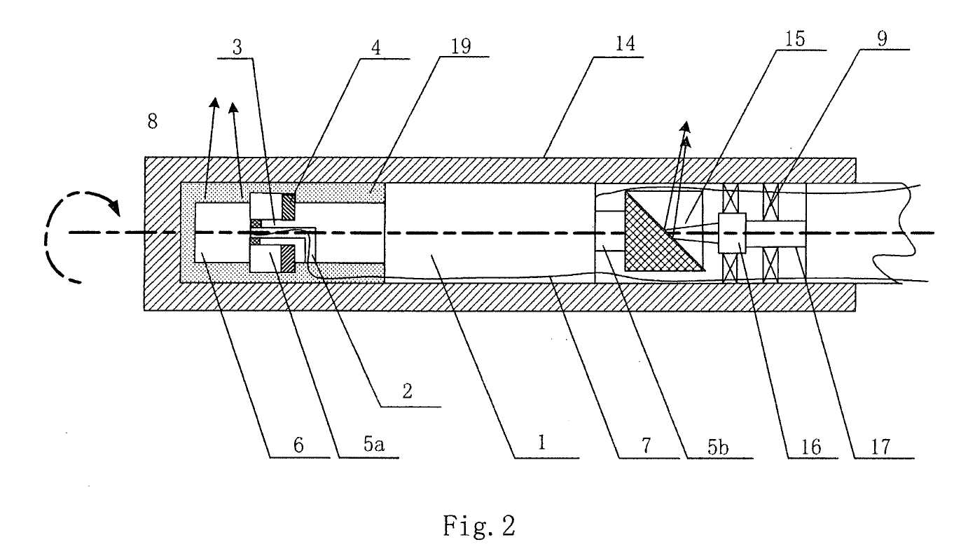

As shown in FIG. 2, a micro ultrasonic motor stator 1, whose shaft 3 connects to a friction layer 4 and magnetic rotors 5a/5b, has a wire channel 2 in it. The friction layer 4 is fixed on the micro ultrasonic motor stator 1 which is riveted with packaging case 14. The rotor 5a/5b is in connection with the shaft 3 by axletree. An ultrasonic transducer 6 and a prism 15 are adhered to the rotor 5a and 5b respectively. A cable 7 connects to the ultrasonic transducer 6 through the wire channel 2 in a probe 8. The ultrasonic transducer 6 is immersed in an acoustic couplant 19. A prism 15, a grim lens 16 and a fiber 17 together make the OCT imaging system, in which the grim lens 16 and fiber 17 are pegged with the packaging case 14 by several carriers 9.

An ultrasonic signal is coupled to the ultrasonic transducer 6 through the cable 7 and is changed into a supersonic wave. By continuously rotating shaft 3 by the micro ultrasonic motor stator 1, a friction generated by the friction layer 4 rotates the rotor 5a and changes the direction of radiation of supersonic wave, so that the radial scan centering on the longitudinal direction of the probe 8 is performed. At the same time, the rotor 5b moves the direction of radiating light and signal light received by radial scan centering on the longitudinal direction of probe 8 and is collected by the prism 15 into the fibre through the grim lens 16.

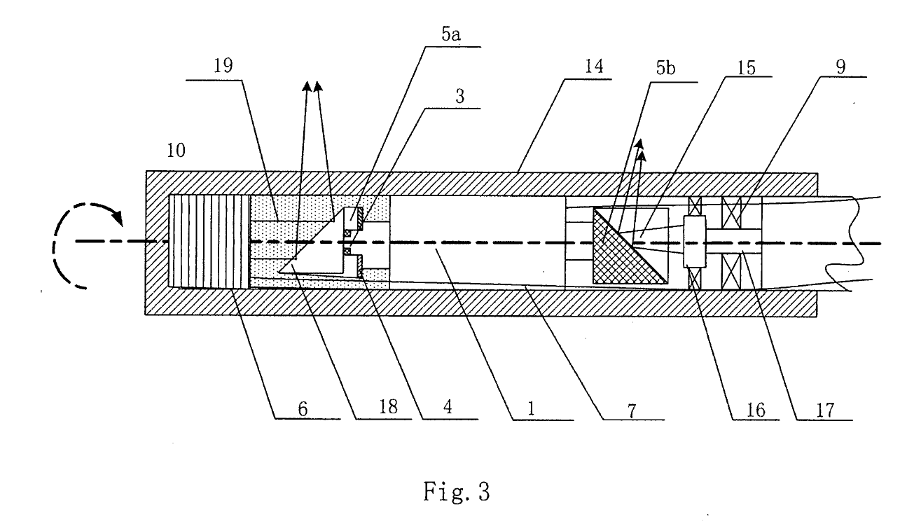

As shown in FIG. 3, a friction layer 4 is fixed on the micro ultrasonic motor stator 1 whose shaft 3 connects to the friction layer 4 and magnetic rotors 5a/5b. The rotor 5a/5b is in connection with the shaft 3 by axletree. Acoustic reflector 18 and prism 15 are adhered to rotors 5a and 5b respectively. The micro ultrasonic motor stator 1 and ultrasonic transducer 6 are fixed with packaging case 14. A cable 7 is coupled to the ultrasonic transducer 6 through a probe 8. The ultrasonic transducer 6 and acoustic reflector 18 are immersed in acoustic couplant 19. Prism 15, grim lens 16 and fiber 17 together make the OCT imaging system, in which the grim lens 16 and the fiber 17 are pegged with packaging case 14 by several carriers 9.

An ultrasonic signal is coupled to the ultrasonic transducer 6 through the cable 7 and is changed into a supersonic wave. By continuously rotating shaft 3 by the micro ultrasonic motor stator 1, friction generated by the friction layer 4 rotates the rotor 5a and changes the direction of acoustic reflector 18, thus changing the direction of emissive and return supersonic waves and achieving the performance of radial scan centering on the longitudinal direction of the probe 10. At the same time, the rotor 5b moves the direction of radiating light and signal light received by radial scan centering on the longitudinal direction of probe 10 and is collected by the prism 15 into fibre through the grim lens 16.

### Application

### EXAMPLE 1

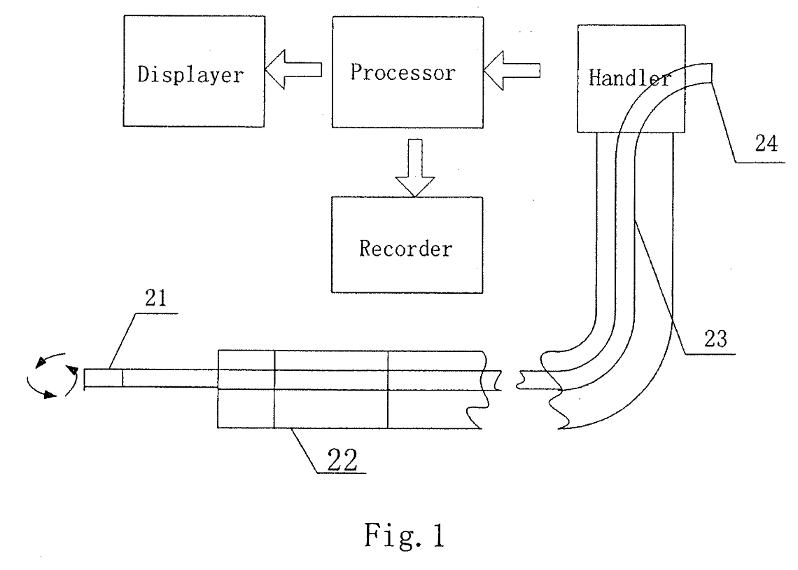

FIG. 1 is the structure of an endoscopic ultrasonic OCT imaging system. Micro ultrasonic OCT probe 21 includes micro ultrasonic transducer, OCT implementation and ultrasonic motor. The micro ultrasonic OCT probe is inserted into a body from biopsy orifice 24 of endoscope 22 through biopsy channel 23, and its rotating part is driven by ultrasonic motor rotor to scan.

### EXAMPLE 2

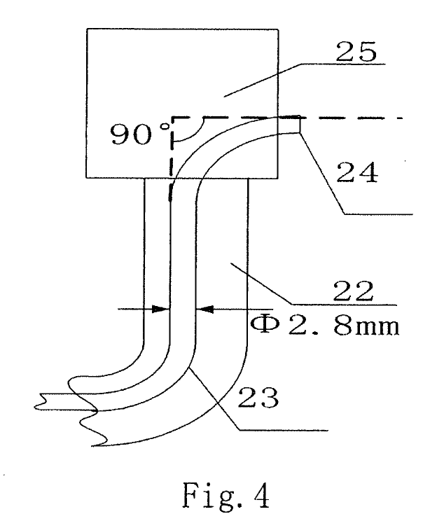

A digestive tract endoscope for checking the digestive tract. An endoscopic ultrasonic OCT probe is inserted into a body through biopsy channel of a digestive tract endoscope. FIG. 4 shows biopsy channel 23 of endoscope 3. It is a Teflon soft pipe with an inner radius of 2.8 mm. As biopsy channel having an orthogonal hard-tube bending below entrance 24, the diameter of ultrasonic OCT probe must be equal to or smaller than 2 mm, and the length of the inflexible part must be less than 12 mm, in order to make sure that the probe can pass.

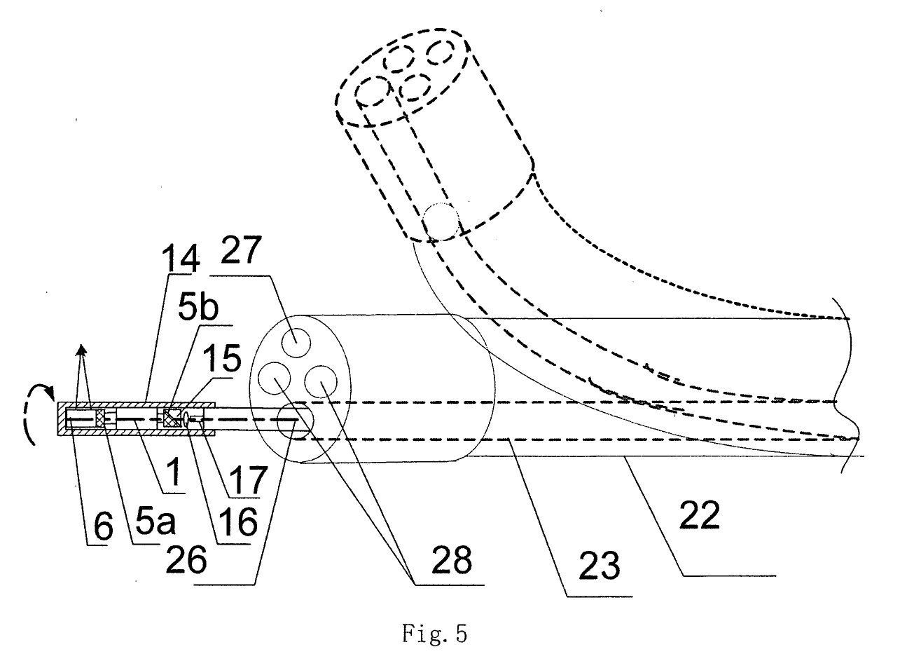

FIG. 5 shows the front part of the digestive tract endoscope. The front part of the endoscope has four holes. Biopsy orifice 26 is for biopsy devices and micro ultrasonic probe scan; CCD (charge-coupled device) imaging implementation 27 is for recording color video image of tissue surface; illuminating channel 28 is for background illuminating. The digestive tract endoscope is of soft-tube lens.

The present invention has following advantage:

(1) Triple diagnosis. Not only can it observe the pathologic changes on the surface of mucosa through endoscope, but can also get the histological tomogram of an organ through OCT scan and ultrasonic scan, thus broadening the diagnosis range and increasing the diagnosis ability of endoscopes.

(2) Long life-time. The biggest drawback of the existing technology is its short life-time. In the existing systems, the motor is placed at the rear end of the endoscope far away from the probe and drives probe only through soft wires. Since the soft wires are easy to damage, the probe itself must be discarded before its life expectancy. On the other hand, the probe of this invention is driven by a micro motor directly mounted on the front end of the probe, and does not need soft wires. Compared with the existing technology, its lifespan is greatly expanded.

(3) Versatility. The ultrasonic OCT endoscope imaging system uses the regular endoscope as a carrier and therefore no special endoscope carrier is required. The system can be used with any endoscope system having a standard biopsy channel for ultrasonic diagnosis and OCT diagnosis, and therefore has wide applications.

(4) Easy operation. When using the endoscope for diagnosis or treatment, the operators, assistants and other medical professionals can operate the system with a monitor, which makes the cooperation between them harmonious and safe. Therefore the system is flexible, convenient and easy to operate.

(5) The driving part has no electromagnetic interference.

1. A micro medical-ultrasonic endoscopic OCT probe comprising:
a micro ultrasonic motor stator for connecting a friction layer and a magnetic rotor;
an ultrasonic transducer and a prism being adhered to a rotor respectively;
an acoustic couplant for immersing the ultrasonic transducer; and
an OCT imaging system comprises a prism, grim lens and a fiber.

2. The micro medical-ultrasonic endoscopic OCT probe of claim 1, further comprising a wire channel, for connecting cable to the ultrasonic transducer.

3. The micro medical-ultrasonic endoscopic OCT probe of claim 1, wherein said friction layer is being fixed on the micro ultrasonic motor stator.

4. The micro medical-ultrasonic endoscopic OCT probe of claim 1, wherein said rotor is connected to a shaft by an axletree.

5. The micro medical-ultrasonic endoscopic OCT probe of claim 1, wherein said grim lens and fiber is being pegged with a packaging case by several carriers.

6. A micro medical-ultrasonic endoscopic OCT probe comprising:
a micro ultrasonic motor stator for connecting a friction layer and a magnetic rotor;
an ultrasonic reflector and a prism being adhered to a rotor respectively;
an acoustic couplant for immersing the ultrasonic transducer; and
an OCT imaging system comprises a prism, grim lens and a fiber.

7. The micro medical-ultrasonic endoscopic OCT probe of claim 6, further comprising a cable coupling to the ultrasonic transducer through the probe.

8. The micro medical-ultrasonic endoscopic OCT probe of claim 6, wherein said friction layer is being fixed onto the micro ultrasonic motor stator.

9. The micro medical-ultrasonic endoscopic OCT probe of claim 6, wherein said rotor is connected to a shaft by an axletree.

10. The micro medical-ultrasonic endoscopic OCT probe of claim 6, wherein said grim lens and fiber is being pegged with a packaging case by several carriers.
